(19) United States
(12) Patent Application Publication
(10) Pub. No.: US 2007/0086911 A1
(43) Pub. Date: Apr. 19, 2007

(54) Disinfection system

(75) Inventors: Atsushi Yamazaki, Kanagawa (JP); Shinichiro Fukui, Kanagawa (JP); Satoshi Arakawa, Kanagawa (JP); Yuichi Hosoi, Kanagawa (JP); Hiroaki Yasuda, Kanagawa (JP)

Correspondence Address: BIRCH STEWART KOLASCH & BIRCH, PO BOX 747, FALLS CHURCH, VA, 22040-0747, US

(73) Assignee: FUJI PHOTO FILM CO., LTD., Minami-Ashigara-shi (JP)

(21) Appl. No.: 11/528,403

(22) Filed: Sep. 28, 2006

(30) Foreign Application Priority Data
Sep. 30, 2005 (JP) 2005-288834

(51) Int. Cl. A61L 2/04

(52) U.S. Cl. 422/1

(57) ABSTRACT

According to the present invention, there is provided a disinfection system for a radiation image conversion panel, a radiation image conversion film, and/or a light shielding bag that can be used to wrap such a radiation image conversion film therein including a disinfection unit which applies at least a disinfection treatment to a radiation image conversion panel, and an image reading apparatus including the disinfection system. In the disinfection system for a radiation image conversion panel, preferably the disinfection treatment by the disinfection unit is at least one of a heat treatment, an ultraviolet irradiation treatment, a chemical application treatment or a gas treatment.

### CROSS-REFERENCE TO RELATED APPLICATION

This application claims priority under 35USC 119 from Japanese Patent Application No. 2005-288834, the disclosure of which is incorporated by reference herein.

### BACKGROUND OF THE INVENTION

1. Field of the Invention

The present invention relates to a disinfection system for a radiation image conversion panel utilizing a stimulable phosphor, a radiation image conversion film using a stimulable phosphor, and/or a light shielding bag that can be used to wrap such a radiation image conversion panel or radiation image conversion film therein.

2. Description of the Related Art

Recently, with a growing interest in measures against infectious diseases among medical professionals, there is desired an apparatus which disinfects a radiation image conversion panel. Moreover, particularly, since a radiation image conversion panel or radiation image conversion film for dental application is handled in the mouth, a likelihood where body fluid such as a saliva of a patient is adhered thereto enhances this desire.

As a disinfection apparatus used for medical instruments, there is proposed an apparatus which disinfects by using an oil of a high temperature (for example, refer to Japanese Patent Application Laid-Open (JP-A) No. 2005-131359). However, the apparatus has a safety issue since an oil is used, and it is unsuitable for disinfecting a radiation image conversion panel that is easily deformed in a structure of the apparatus.

Consequently, in practice, disinfection is performed by wiping with alcohol such as ethanol. As a result, there is a problem in that disinfection becomes uneven, incomplete, and inefficient, since disinfection is manually performed one by one.

### SUMMARY OF THE INVENTION

The present invention has been made in view of the above circumstances and provides a disinfection system.

A first aspect of the present invention provides an image reading apparatus comprising a disinfection system which applies a disinfection treatment to a radiation image conversion panel, radiation image conversion film, and/or a light shielding bag that can be used to wrap such a radiation image conversion panel or radiation image conversion film therein.

A second aspect of the present invention provides a disinfection system for a radiation image conversion panel comprising a disinfection unit which applies a disinfection treatment to at least a radiation image conversion panel, a radiation conversion film, and/or a light shielding bag that can be used to wrap such a radiation image conversion panel or radiation image conversion film therein.

### BRIEF DESCRIPTION OF THE DRAWINGS

FIG. 1 is a block diagram of the inside of an image reading apparatus comprising a disinfection system.

FIG. 2 is a diagram of the outside of an image reading apparatus comprising a disinfection system.

FIG. 3 is a diagram of the inside of an image reading apparatus comprising a disinfection system.

FIG. 4 is a diagram of the inside of an image reading apparatus comprising a disinfection system.

FIG. 5 is a diagram of the inside of an image reading apparatus comprising a disinfection system.

### DETAILED DESCRIPTION OF THE INVENTION

[Disinfection System of Radiation Image Conversion Panel]

The disinfection system for a radiation image conversion panel of the present invention comprises a disinfection unit which applies a disinfection treatment to at least a radiation image conversion panel, a radiation conversion film, and/or a light shielding bag that can be used to wrap such a radiation image conversion panel or radiation image conversion film therein (referred to sometimes below as “items to be disinfected”). The disinfection system of the present invention may be incorporated in an image reading apparatus, or may be used as an apparatus independent to the apparatus.

The disinfection treatment by the disinfection unit is preferably, from a practical viewpoint, at least a treatment selected from a heat treatment, an ultraviolet irradiation treatment, a chemical application treatment, a gas treatment, with heat treatment and ultraviolet irradiation being more preferable. If the disinfection treatment is a heat treatment, the temperature of the heat treatment is preferably 60° C. to 200° C., and more preferably 90 to 120° C. Moreover, the time for the heat treatment is preferably 1 second to 10 minutes, and more preferably 10 seconds to 5 minutes.

For example, in order to kill botulinum toxins it is possible to carry out heat treatment with heating to 120° C. for about 30 minutes.

If disinfection is performed by the heat treatment, the disinfection unit preferably comprises a temperature control unit. As to the temperature control unit, a normal temperature control device may be used. By providing the temperature control unit, the items for disinfection can be set within the abovementioned temperature range.

The heating unit is not specifically limited and may be for example a unit which supplies hot air to the radiation image conversion panel, or a unit using an infrared heater or a far infrared heater. However, from the viewpoint of temperature controllability and safety, the heat treatment is preferably performed using either one of an infrared heater and a far infrared heater. The power when using the infrared heater or the far infrared heater is preferably 50 to 1000 W. As another way to carry out heat treatment, microwaves can be used. In this case, at least waves in the frequency range 300 MHz to 30 GHz should be included. Using microwaves is highly safe, and the speed of heating is fast and heating efficiency high. There is also the merits that it is possible to uniformly heat complicated shaped objects and the operation and control thereof is simple.

Moreover, the ultraviolet irradiation unit for the disinfection treatment is a unit which irradiates ultraviolet light by an ultraviolet lamp onto the radiation image conversion panel. However, if ultraviolet light is over-irradiated, the phosphor layer might be sensitized. Therefore it is necessary to appropriately adjust the irradiation time. With the use of ultraviolet light irradiation there are the merits that the operation thereof is simple, it is possible to maintain a hygienic environment, and it is highly safe.

The irradiation energy of ultraviolet light in the disinfection process is preferably 0.04 J/cm² or above, and a range of 0.04 J/cm² to 40 J/cm² is more preferable. If the energy is more than 0.04 J/cm² then effective disinfection can be carried out. Further, it is preferable to include wavelengths at least in the range of 250 to 280 nm. In particular it is preferable to include the wavelength 254 nm, known as the wavelength with the strongest disinfecting power. Further, when considering the prevention of ultraviolet light fogging during erasing, it is preferable to carry out the processing for erasing of the image data using eraser unit 39 after carrying out the disinfection treatment.

A first embodiment of chemical application treatment serving as another unit of the disinfection unit, includes providing an immersion tank filled with an agent, and a treatment of immersing the item to be disinfected into the immersion tank. In the case of the immersion treatment, the immersion time is preferably about 1 to 600 seconds, and the immersion may be performed appropriately for a plurality of times. In addition to the immersion treatment, a unit which spray-coats an agent may be employed. The agent includes: alcohol such as ethanol; aldehyde such as glutaraldehyde; and peracetic chlorine.

Further, as an aspect of a second embodiment of chemical application treatment is where an agent is applied by passing the item to be disinfected between a pair of rollers impregnated with one of the above agents. Plural pairs of the rollers may be arranged either in series or arranged intermittently.

For disinfection using a gas (gas treatment), ethylene oxide, ozone can be blown onto the items to be disinfected. It is possible to carry out processing using ethylene oxide at a temperature close to room temperature. Ozone can demonstrate excellent effects in breaking down germs and organic matter, because of its strong oxidizing power.

Other examples of disinfecting methods can be given. These include the irradiation of electromagnetic waves and rays with wavelengths below that of the ultraviolet region, such as γ-rays and X-rays, onto the items to be disinfected. These methods are particularly effective when the carrying out of heat treatment is difficult.

The disinfection system of the present invention preferably comprises an image reading unit which reads out an image on the radiation image conversion panel and/or the radiation image conversion film. The image reading unit provides an advantage in that the disinfection treatment and the image reading treatment can be realized in one system.

From the viewpoint of protecting the phosphor layer, the radiation image conversion panel and/or the radiation image conversion film may be formed with a protective layer. If the radiation image conversion panel formed with the protective layer is subjected to a disinfection treatment by means of heating, it may be deformed and thus becomes deficient depending on its material. Consequently, if such a disinfection treatment by means of heating is applied, the thermal shrinkage rate (JISC2151 which is incorporated herein by reference, at 150° C. for 30 minutes) of the protective layer is preferably 1% or less, and more preferably 0.01 to 0.8%. If the thermal shrinkage rate is 1% or less, the deformation due to thermal shrinkage can be prevented.

The protective layer of the radiation image conversion panel and/or radiation image conversion film is preferably subjected to a heat treatment of 60° C. or more, at least either before or at the time of its formation. By applying such a heat treatment, the deformation due to heating can be prevented even if the disinfection treatment by means of heating is performed.

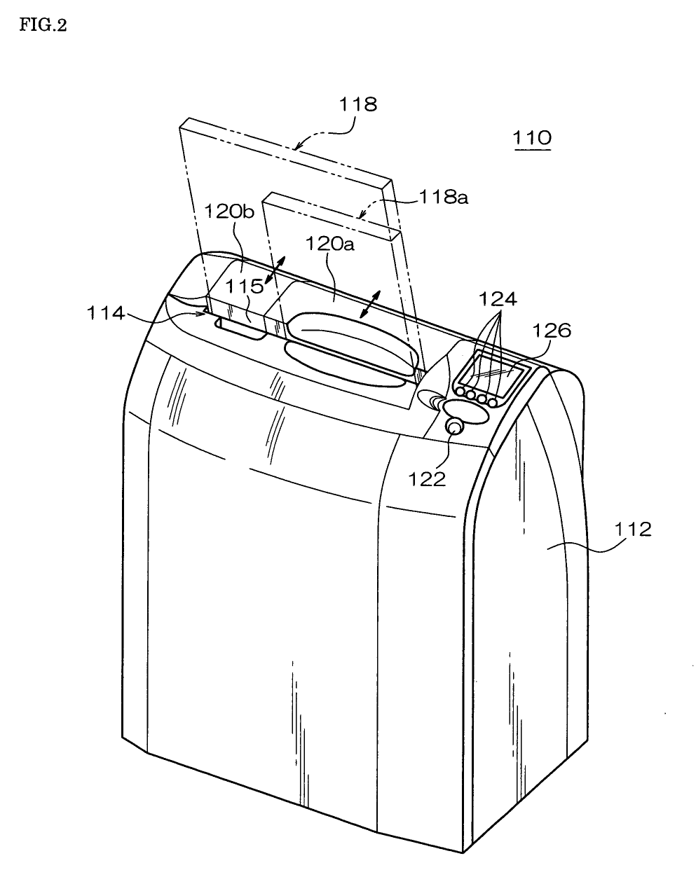

Radiation image conversion films are generally films of approximately 3 cm×4 cm of a form which can be used in the taking of dental internal oral X-ray images. Light shielding bags that can be used to wrap such radiation image conversion films are light shielding bags of about the same size for wrapping radiation image conversion films therein, and after wrapping they can be sealed with double-sided tape or the like to give a sealed envelope state. Further, examples of possible embodiments are disclosed in the Examples and FIGS. 2 to 4 of Japanese Patent Application Laid-Open No. S64-49032 or Japanese Patent Application Publication (JP-B) No. 6-100791.

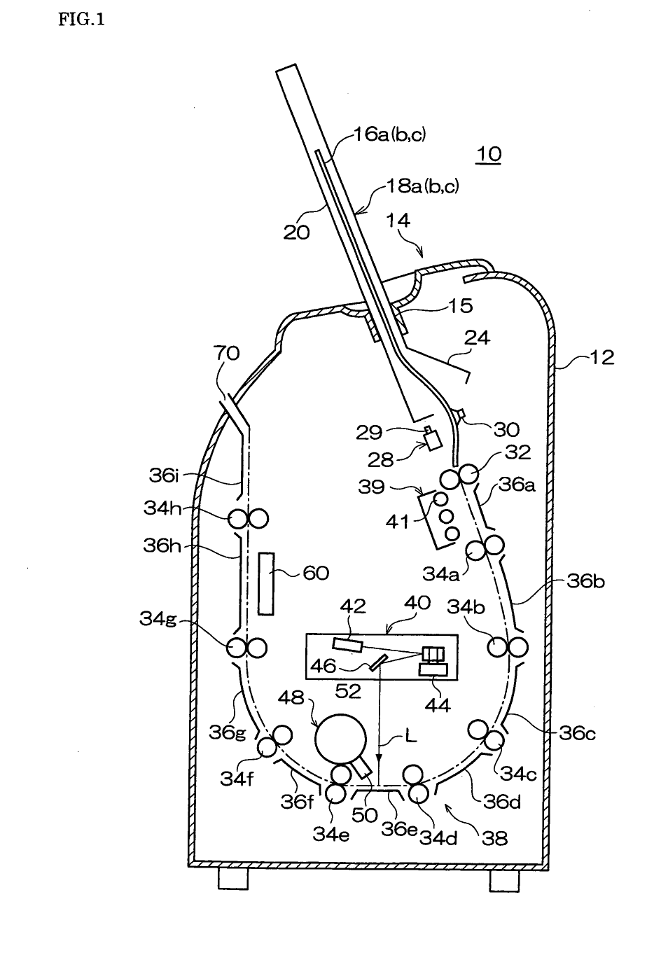

Next is a description of the image reading apparatus comprising the disinfection system of the present invention, with reference to FIG. 1.

The image reading apparatus 10 comprises a cassette loading portion 14 on the top of a casing 12. Through a loading aperture 15 formed in this cassette loading portion 14, is loaded an image recording medium having radiation image data cumulatively recorded therein, such as a cassette 18a (18b, 18c) housing an image conversion panel 16a (16b, 16c). In a case of a radiation image conversion panel used for dental application, the cassette may not be used in some cases. Specifically, a radiation image conversion panel stored in a predetermined bag is taken out and subjected to various treatments.

The width of the cassette 18b is narrower than that of the cassette 18a. The width of the cassette 18c is narrower than that of the cassette 18b. The width of the radiation image conversion panel 16b stored in the cassette 18b is narrower than that of the radiation image conversion panel 16a stored in the cassette 18a. The width of the radiation image conversion panel 16c stored in the cassette 18c is narrower than that of the radiation image conversion panel 16b stored in the cassette 18b.

In the description hereunder, although the cassette 18a and the radiation image conversion panel 16a are used, the description is similarly applied to the cassettes 18b and 18c and the radiation image conversion panels 16b and 16c.

The cassette 18a comprises a mainframe 20 which houses the radiation image conversion panel 16a, and a lid member 24 which forms an opening 22 for putting in/taking out the radiation image conversion panel 16a.

In the vicinity of the loading aperture 15 inside of the image reading apparatus 10 is arranged: a lock release mechanism 28 which releases locking of the lid member 24 of the cassette 18a; a suction cup 30 which attracts the radiation image conversion panel 16a and takes it out from the cassette 18a with the lid member 24 open; and a roller pair 32 which interposes therebetween the radiation image conversion panel 16a that has been taken out by the suction cup 30, and conveys it. The lock release mechanism 28 has a lock release pin 29 for releasing a cassette lock unit (not shown) that is inserted into the cassette 18a.

Lined up with the roller pair 32, a plurality of carrier roller pairs 34a to 34h and a plurality of guide plates 36a to 36i are arranged, constituting a curved carrier track 38. Between the roller pair 32 and the carrier roller pair 34a is arranged an eraser unit 39 for erasing the radiation image data remaining on the radiation image conversion panel 16a having the read processing completed. The eraser unit 39 has an erase light source 41 such as a cold-cathode tube which outputs erase light.

In the approximate center of the image reading apparatus 10 is arranged a scan unit 40 which emits laser beams L serving as exciting light and scans the radiation image conversion panel 16a. The scan unit 40 comprises: a laser oscillator 42 which outputs a laser beam L; a polygon mirror 44 serving as a rotating polygon mirror which deflects the laser beam L in the main scanning direction of the radiation image conversion panel 16a; and a reflection mirror 46 which reflects the laser beam L to guide to the radiation image conversion panel 16a passing through on the guide plate 36e.

Between the carrier roller pair 34e and the scan unit 40 is arranged a reading unit 48. The reading unit 48 comprises: a condensing guide 50 having one end arranged in the vicinity of the radiation image conversion panel 16a on the guide plate 36e; and a photomultiplier 52 which is connected to the other end of the condensing guide 50, and converts stimulable luminous light obtained from the radiation image conversion panel 16a into electric signals.

Moreover, between carrier roller pairs 34e and 34h is provided a disinfection unit 60. Here, the radiation image conversion panel applied with the disinfection treatment is conveyed to the outlet 70 and taken out.

The image reading apparatus comprising the disinfection unit 60 operates as described below. Firstly, the cassette 18a which houses the radiation image conversion panel 16a having the radiation image data recorded therein, is supplied to the image reading apparatus 10. The cassette 18a is loaded into the loading aperture 15 of the cassette loading portion 14 having the lid member 24 faced downward. The locking of the lid member 24 is released through the lock release mechanism 28.

Next, the radiation image conversion panel 16a in the cassette 18a is taken out from the cassette 18a under the suction effect of the suction cup 30. The tip of the radiation image conversion panel 16a that has been taken out from the cassette 18a is interposed between the roller pair 32, and at the same time the attraction and the holding of the radiation image conversion panel 16a by the suction cup 30 are released.

As a result, the radiation image conversion panel 16a is conveyed vertically downward under the rotation effect of the roller pair 32. This radiation image conversion panel 16a is conveyed by the curved carrier track 38 comprising the carrier roller pairs 34a to 34h and the guide plates 36a to 36i.

When the carrier roller pairs 34b and 34c are synchronously driven and thereby the radiation image conversion panel 16a is conveyed to a pull-over device 54, the radiation image conversion panel 16a is released from being interposed between the carrier roller pair 34b and 34c.

The radiation image conversion panel 16a having the pull-over processing completed as described above, is conveyed for sub-scanning between the carrier roller pairs 34d and 34e, and the laser beam L emitting from the scan unit 40 scans over the radiation image conversion panel 16a in the main scanning direction orthogonal to the sub-scanning direction. That is, the laser beam L output from the laser oscillator 42 is reflected and deflected by the polygon mirror 44 which rotates at high speed, and is then guided to the radiation image conversion panel 16a through the reflecting mirror 46.

On the other hand, the radiation image conversion panel 16a irradiated with the laser beam L outputs stimulable luminous light corresponding to the cumulatively recorded radiation image data. This stimulable luminous light is guided to the photomultiplier 52 constituting the reading unit 48 through the condensing guide 50 that is arranged in the vicinity along the main scanning direction of the radiation image conversion panel 16a.

The radiation image conversion panel 16a in which the radiation image data has been read out in this manner, is disinfected by the disinfection unit and conveyed to the carrier roller pair 34h side. Then, it is conveyed to the outlet 70 and taken out. If the disinfection unit controls the temperature by the temperature control unit in the heat treatment unit, the surface temperature measurement method when the temperature is controlled, is preferably performed by bringing the radiation image conversion panel into contact with a thermocouple.

The disinfected radiation image conversion panel 16a that has been taken out, is supplied for image capturing of the next radiation image data.

In addition to the above, aspects of embodiments of the disinfection systems of the present invention are such as the following.
(1) A first embodiment is an embodiment in which the radiation image conversion panel or radiation image conversion film which has had images taken thereon is wrapped within a light shielding bag, and this sealed. In this sealed state it is conveyed to the disinfection unit of the disinfection system, and here disinfection treatment is carried out. Specifically, the light shielding bag is conveyed to the disinfection unit of the disinfection system using conveyance rollers, and here the disinfection treatment is carried out by the irradiation of ultraviolet light from an ultraviolet light source. After this, the light shielding bag is conveyed by rollers to a light shielding bag opening unit. While one edge of the light shielding bag is held down by a holding member the other end of the light shielding bag is opened by use of an opening means such as a cutter or the like, and the radiation image conversion film is taken out, and appropriately conveyed to the image reading unit. The light shielding bag from which the radiation image conversion film has been removed is disposed of appropriately.

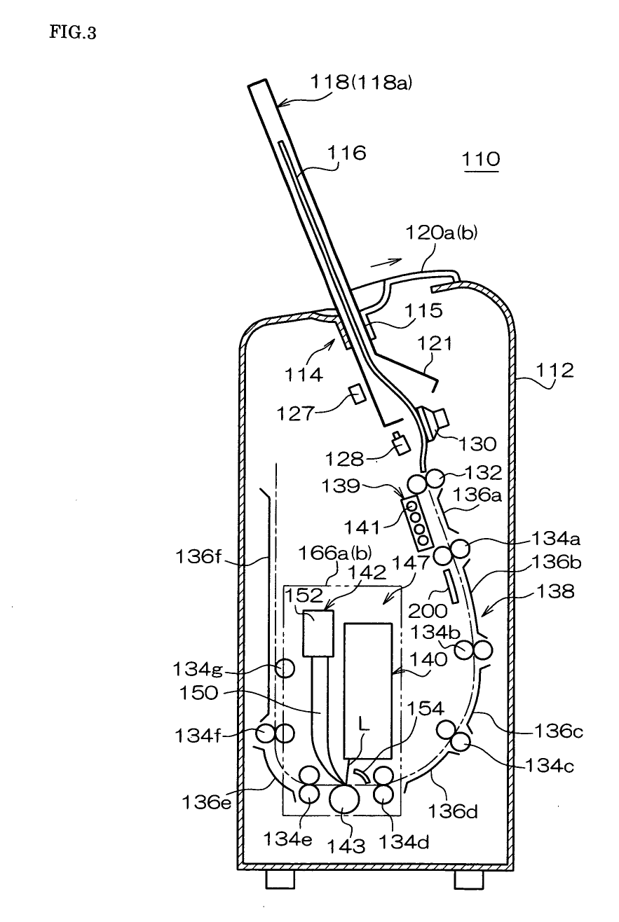

By this embodiment it is possible to disinfect in a sealed condition, and avoid adherence of bodily fluids or germs to the radiation image conversion panel or radiation image conversion film when the light shielding bag is opened. The disinfection treatment of the disinfection unit can be carried out, as described above, by heat treatment, chemical application treatment, gas disinfection treatment (as is also the case in the embodiments that follow).
(2) A second embodiment is an embodiment in which the radiation image conversion film before it has had images taken thereon is wrapped within a light shielding bag, and this sealed. In this sealed state it is conveyed to the disinfection unit of the disinfection system, and here disinfection treatment is carried out. Specifically, the light shielding bag is conveyed to the disinfection unit of the disinfection system using conveyance rollers, and here heat treatment (disinfection treatment) is carried out by a heater. After this, the light shielding bag is discarded. By this embodiment it is possible, because disinfection is completed in the sealed condition, it can be loaded into the mouth or into the body of a patient just as it is.
(3) A third embodiment is an embodiment in which the radiation image conversion panel and/or radiation image conversion film is conveyed to the disinfection unit of the disinfection system, and here disinfection treatment is carried out. Specifically, radiation image conversion panel and/or radiation image conversion film is conveyed to the disinfection unit using conveyance rollers, and here the radiation image conversion panel and/or radiation image conversion film is passed through the nip of a pair of sponge rollers impregnated with an agent, thereby carrying out disinfection treatment. After this, appropriate conveyance thereof is made to the image reading unit. By this embodiment it is possible to disinfect body fluids and germs that have adhered when removing from the light shielding bag, and thereby avoid contagion from the radiation image conversion panel or radiation image conversion film.
(4) A fourth embodiment will now be explained with reference to FIGS. 2 and 3. In FIG. 2, a view is shown of the external appearance a radiation image information reading device 110 common to embodiments 4 to 6, in FIG. 3 the internal structure is shown.

In FIG. 2, the radiation image information reading device 110 is provided with a cassette loading portion 114 at the top portion of casing 112, and the cassette 118 (118a) containing the radiation image conversion panel with the radiation image information stored and recorded thereon is loaded into the loading aperture 115 formed in the cassette loading portion 114. The cassette 118a is smaller in size that the cassette 118.

The radiation image conversion panel is a panel having a storage phosphor layer which, when irradiated with radiation (X-rays, α-rays, β-rays, γ-rays, electron beams, ultraviolet rays or the like) a portion of the radiation energy is stored, and then afterwards, with the irradiation by excitation light, of laser light or visible light and the like, stimulated phosphorescence in response to the stored energy is displayed. When the remaining energy is erased by irradiation with erasing light including light in the wavelength of the excitation light of the phosphor, the panel can be reused.

The cassette loading portion 114 has cover portion members 120a, 120b which are independently displaceable in the direction of the arrow. When the large size cassette 118 is loaded, the cover portion members 120a, 120b both displace and the entire loading aperture 115 is opened. When the small cassette 118a is loaded, only the cover portion member 120a displaces and a portion of the loading aperture 115 is opened. By this arrangement, the ingression of dust into the inner portion of the radiation image information reading device 110 can be repressed. On a side portion of the cassette loading portion 114, a power source button 122, an operating button 124, a display portion 126 and the like are disposed.

In FIG. 3, at an internal portion of the radiation image information reading device 110 near to the loading aperture 115, there is: a panel information readout portion 127 for reading out various information, such as the size, sensitivity and the like, identification number and the like (referred to as “panel information” below) of the radiation image conversion panel 116 accommodated in the loaded cassette 118 (118a); a lock release mechanism for releasing the lock of the lid portion member 121 of the cassette 118 (118a); a suction pad 130 for suctioning and taking out the radiation image conversion panel 116 from the cassette 118 (118a) with opened lid portion member 121; and nip rollers 132 for nipping and conveying the radiation image conversion panel 116 that has been taken out by the suction pad 130.

The panel information readout portion 127 configured with a read-out unit, such as a bar code reader, RFID or the like, reads out the panel information recorded on a bar-code, IC chip or the like mounted on the cassette 118 (118a) or the radiation image conversion panel 116.

Plural conveying rollers 134a to 134g and plural guide plates 136a to 136f are disposed in conjunction to the nip rollers 132, and these configure the curved conveying path 138. The curved conveying path 138, after extending in a downward direction from the cassette loading portion 114, becomes substantially horizontal at the lowest portion thereof, then extends substantially vertically upwards. By this configuration the radiation image information reading device 110 can be made compact.

Between the nip rollers 132 and the conveying rollers 134a, an erasing unit 139 is disposed for erasing the radiation image information remaining in the radiation image conversion panel 116 after the read-out process has been completed. The erasing unit 139 has plural erasing light sources 141 made up from cold cathode tubes that emit erasing light.

Disposed between the lowest portion of the curved conveying path 138 and the conveying rollers 134d, 134e, is a platen roller 143. At the upper portion of the platen roller 143, accommodated in a housing 145, is disposed a scanning unit 147 for reading out the radiation image information stored and recorded in the radiation image conversion panel 116.

The scanning unit 147 is provided with: an excitation portion 140, for guiding the light of the excitation light laser beam L, scanning the radiation image conversion panel 116; and an image information read-out portion 142, for reading out the light of the photo-stimulated emission related to the radiation image information that is output from the excitation due to the laser beam L. The image information read-out portion 142 is provided with a photomultiplier 152, for converting the light of photo-stimulated emission obtained from the radiation image conversion panel 116 into an electrical signal, the photomultiplier 152 being connected on one edge portion to a light guide 150 disposed in the vicinity of the radiation image conversion panel 116 above the platen roller 143, and on the other edge portion to light guide 150. In order to increase the collecting efficiency of the accelerated phosphorescent light, a collection mirror 154 is placed in the vicinity of one end of the light guide.

In the fourth embodiment is shown an example of carrying out heat treatment using a heater 200 provided as a disinfection system at the lower side of the erasing unit 139.

The radiation image information reading device 110 of this illustrative embodiment of the invention is basically configured as above, and the operation thereof will now be explained.

First, lid portion member 121 is moved down and the cassette 118 (118a) accommodating the radiation image conversion panel 116 is loaded at the loading aperture 115 of the cassette loading portion 114.

Next, the panel information read-out portion 127 reads out the panel information including the type discriminator of the radiation image conversion panel 116 and the like from the radiation image conversion panel 116 accommodated in the cassette 118 (118a).

When panel information can be read out the lock release mechanism 128 is driven, the locked condition of the lid portion member 121 is released and lid opened. Next, the suction pad 130 suctions the radiation image conversion panel 116, and pulls out the radiation image conversion panel 116 from the cassette 118 (118a) and supplies it between the nip rollers 132. The radiation image conversion panel 116, nipped between the nip rollers 132, is conveyed past the disinfection unit 139, and conveyed to below the lower portion of the scanning unit 147 via the curved conveying path 138 formed from the conveying rollers 134a to 134b and guide plates 136a to 136f.

The radiation image conversion panel 116 is conveyed in a substantially horizontal direction in the sub-scanning direction by the conveying rollers 134d to 134e. Here, the laser beam L emitted from the excitation unit 140 is guided to the radiation image conversion panel 116 supported on the lower face portion by the platen roller 143, and the radiation image conversion panel 116 is scanned in the main direction.

The radiation image information that is stored and recorded in the radiation image conversion panel 116 is excited by the irradiation with the laser beam L, and light of the photo-stimulated emission is output. This light of the photo-stimulated emission is directly illuminated into the lower end portion of the light guide 150 configuring the image information read-out portion 142, disposed adjacent to and along the main scanning direction of the radiation image conversion panel 116, or illuminated into the same via a light-converging mirror 154. The light of the photo-stimulated emission that is illuminated into the light guide 150 is guided to the upper end portion photomultiplier 152, being internally reflected multiple times. The photomultiplier 152 converts the light of the photo-stimulated emission illuminated therein to an electrical signal, and in this way the radiation image information that is stored and stored in the radiation image conversion panel 116 is read out.

Next, the radiation image conversion panel 116 from which the radiation image information has been read out is conveyed from the scanning unit 147 again to the erasing unit 139 side via the curved conveying path 138. Then, the disinfection treatment is carried out by a heater provided at the adjacent side of the erasing unit 139. After the disinfection treatment is carried out the radiation image conversion panel 116 is conveyed to the erasing unit 139.

The erasing unit 139 controls the setting of the erasing light amount of the erasing light sources 141 according to the panel information read out by the panel information read-out portion 127 and the radiation image information read out by the image information read-out portion 142. By the erasing light output from the erasing light sources 141, erasing processing is carried out of the radiation image information that remains in the radiation image conversion panel 116. Reference can be made to the disclosure of Japanese Patent Application Laid-Open (JP-A) No. 11-352615 for methods for the erasing of residual radiation images.

The radiation image conversion panel 116 from which the remaining radiation image information has been erased is accommodated in the cassette 118 (118a) loaded into the cassette loading portion 114, after lid closure with the lid portion member 121, it is removed from the cassette loading portion 114 and can be supplied for the next image exposure.

In the above, description of a case in which read-out of the radiation image information has been made by scanning of the radiation image conversion panel 116 with the laser beam L, however, it is applicable also to, for example, recording image information by scanning a recording medium with a laser beam L modulated according to the image information.

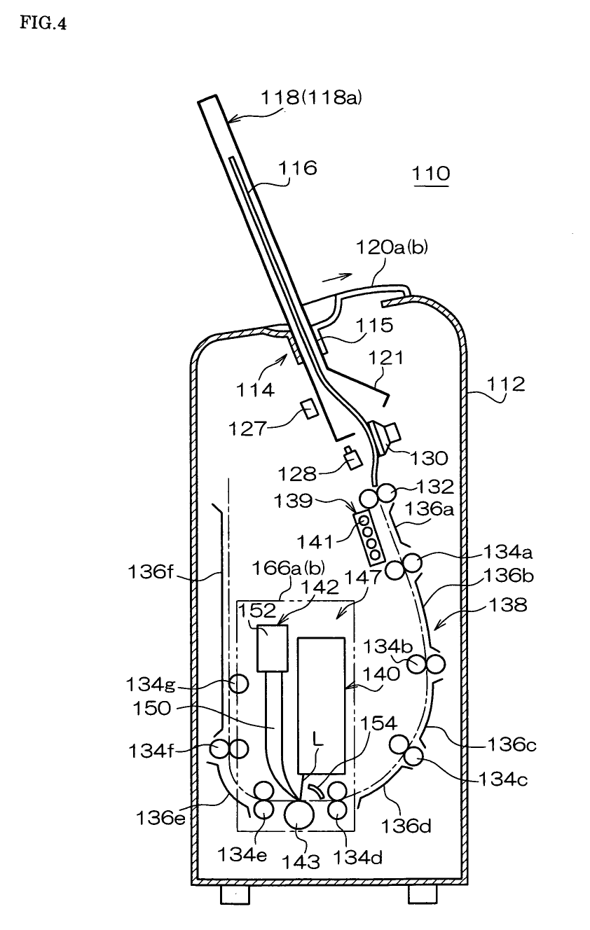

Further, in a fifth embodiment, as is shown in FIG. 4, it is configured so that a heater is not provided and the erasing unit 139 combines the function of the disinfecting system. That is to say this is an embodiment in which, after the reading out of the radiation image information, around the time of the erasing processing of the remaining radiation image information in the radiation image conversion panel 116, or during the processing, disinfection treatment can be carried out by the output of erasing light from the erasing light sources 141. According to the fifth embodiment it is possible to selectively carry out erasing processing and disinfection treatment, giving superior operating characteristics.

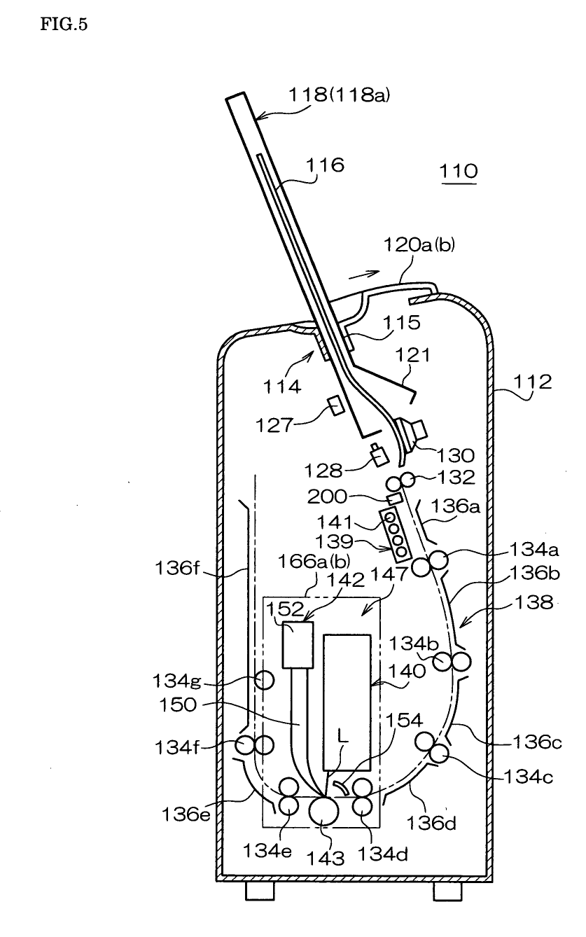

Further, in a sixth embodiment, as is shown in FIG. 5, there is an embodiment in which heat treatment using the heater 200 as the disinfection system of the fourth embodiment is provided further to the upper side than the erasing unit 139. That is to say, after the reading out of the radiation image information, after carrying out the erasing processing on the remaining radiation image information of the radiation image conversion panel 116 by the output of erasing light from the erasing light sources 141, disinfection treatment is carried out by the heater 200.

The radiation image conversion panel and radiation image conversion film applied to the disinfection system of the present invention has a structure, for example where an interlayer, a phosphor layer, a protective layer, and the like are sequentially formed on a support. Hereunder is a description of materials and the like of the respective layers.

(Support)

For the support, a material such as PET, polycycloolefine, PEN (polyethylene naphthalate), PVA (polyvinyl alcohol), a nanoalloy polymer of PET and PEI (polyetherimide), or a transparent aramid is preferably used. In particular, it is desirably a base material having a glass transition temperature (Tg) of 85° C. or more, and preferably 100° C. or more. It is preferably made from a material, such as polycycloolefine, PEN (polyethylene naphthalate), PVA (polyvinyl alcohol), a nanoalloy polymer of PET and PEI (polyetherimide), or a transparent aramid having a glass transition temperature of 85° C. or more. Furthermore, it is more preferably made from a material, such as polycycloolefine, PEN (polyethylene naphthalate), a nanoalloy polymer of PET and PEI (polyetherimide), or a transparent aramid having a glass transition temperature of 100° C. or more.

(Interlayer)

For the interlayer, a transparent high molecular material such as: a cellulose derivative such as acetylcellulose or nitrocellulose; or a synthesized high molecular material of polymethyl methacrylate, polyvinyl butyral, polyvinyl formal, polycarbonate, polyvinyl acetate, vinyl chloride/vinyl acetate copolymer, fluororesin, polyethylene, polypropylene, polyester, acrylic, polyparaxylylene, PET, a hydrochlorinated rubber, a vinylidene chloride copolymer, or the like may be used. These synthesized high molecular materials forming the interlayer may be used as a polymer or a monomer, but are preferably a material which crosslinks by irradiation of heat, visible light, UV light, electron beams, or the like.

If the interlayer is provided on the support, in order to improve the adhesiveness, a coupling agent such as a silane coupling agent and a titanate coupling agent is preferably added. Furthermore, in order to improve the coating property of the interlayer composition and the physical properties of the cured thin film, and to apply a photosensitivity to the coated film, there may be contained various additives for example various polymers and monomers having hydroxyl groups, colorants such as pigments and dyes, a stabilizer such as an anti-yellowing agent, an anti-aging agent, and an ultraviolet absorber, a heat acid generator, a photosensitive acid generator, a surfactant, a solvent, a cross-linking agent, a hardening agent, a polymerization inhibitor, and the like, according to the purpose.

Moreover, in order to improve the durability and to prevent bleeding and unevenness, the interlayer may contain organic or inorganic powder. If the powder is contained, it is preferably about 0.5 to 60% by weight with respect to the weight of the interlayer. The powder is preferably one that has an absorption in a specific bandwidth, such as ultramarine blue, or white powder which does not exhibit a specific absorption in a wavelength region of generally 300 to 900 nm. The volume average particle diameter of the powder is preferably about 0.01 to 10 μm, and more preferably about 0.3 to 3 μm. Generally, the particle size has a distribution, but the distribution is preferably narrow.

(Phosphor Layer)

Preferred examples of the stimulable phosphor used for the phosphor layer include a stimulable phosphor represented by the formula (M_{I-f}·M_{f}^{I})X·bM^{III}X₃″:cA (formula (I)) described in JP-A No. 7-84588. From the standpoint of stimulable luminescent brightness, M^{I} in the formula (I) is preferably Rb, Cs, and/or Cs-containing Na or Cs-containing K, and particularly preferably at least one of alkali metals selected from Rb and Cs. M^{III} is preferably at least one of trivalent metals selected from Y, La, Lu, Al, Ga, and In. X″ is preferably at least one of halogens selected from F, Cl, and Br. The b value expressing the rate of content of M^{III}X₃″ is preferably selected from a range of 0≦b≦10⁻².

In the formula (I), the activator A is preferably at least one of metal selected from Eu, Th, Ce, Tm, Dy, Ho, Gd, Sm, Tl, and Na, and particularly preferably at least one of metal selected from Eu, Ce, Sm, Tl, and Na. Moreover, the C value expressing the amount of activator is preferably selected from a range of 10⁻⁶<C<0.1, from the point of stimulable luminescent brightness.

Moreover, the following stimulable phosphors may be used: SrS:Ce, Sm, SrS:Eu, Sm, ThO₂:Er, and La₂O₂S:Eu, and Sm, described in U.S. Pat. No. 3,859,527;

ZnS:Cu, Pb, BaO·xAl₂O₃:Eu (wherein 0.8≦x≦10), and M^{III}O·xSiO₂:A (wherein: M^{II} is Mg, Ca, Sr, Zn, Cd, or Ba; A is Ce, Tb, Eu, Tm, Pb, Tl, Bi, or Mn; and x is 0.5≦x≦2.5) described in JP-AJP-A No. 55-12142;

(Ba_{1-x-y}, MgX, Cay) FX:aEu²⁺(wherein: X is at least one of Cl and Br; x and y is 0≦x+y≦0.6; and xy≠0, and a is 10⁻⁶≦a≦5×10⁻²) described in JP-A No. S55-12143;

LnOX:xA (wherein: Ln is at least one of La, Y, Gd, and Lu; X is at least one of Cl and Br; A is at least one of Ce and Tb; and x is 0<x<0.1) described in JP-AJP-A No. 55-12144;

(Ba₁₋ₓ, M²⁺X) FX:yA (wherein: M is at least one of Mg, Ca, Sr, Zn, and Cd; X is at least one of Cl, Br, and I; A is at least one of Eu, Tb, Ce, Tm, Dy, Pr, Ho, Nd, Yb, and Er; x is 0≦x≦0.6; and y is 0≦y≦0.2) described in JP-AJP-A No. 55-12145;

phosphors represented by the composition formula of M^{II}FX·xA:yLn (wherein: M^{II} is at least one of Ba, Ca, Sr, Mg, Zn, and Cd; A is at least one of BeO, MgO, CaO, SrO, BaO, ZnO, Al₂O₃, Y₂O₃, La₂O₃, In₂O₃, SiO₂, TiO₂, ZrO₂, GeO₂, SnO₂, Nb₂O₅, Ta₂O_{5, and ThO}₂; Ln is at least one of Eu, Th, Ce, Tm, Dy, Pr, Ho, Nd, Yb, Er, Sm, and Gd; X is at least one of Cl, Br, and I; and x and y are respectively 5×10⁻⁵≦x≦0.5 and 0≦y≦0.2) described in JP-AJP-A No. 55-160078;

phosphors represented by the composition formula of (Ba₁₋ₓ, M^{II}ₓ) F₂·aBaX₂:yEu, zA (wherein: M^{II} is at least one of beryllium, magnesium, calcium, strontium, zinc, and cadmium; X is at least one of chlorine, bromine, and iodine; A is at least one of zirconium and scandium; and a, x, y, and z are respectively 0.5≦a≦1.25, 0≦x≦1, 10⁻⁶≦y≦2×10⁻¹, and 0<Z≦10⁻²) described in JP-AJP-A No. 56-116777;

phosphors represented by the composition formula of (Ba₁₋ₓ, M^{II}ₓ)F₂·aBaX₂:yEu, zB (wherein: M^{II} is at least one of beryllium, magnesium, calcium, strontium, zinc, and cadmium; X is at least one of chlorine, bromine, and iodine; and a, x, y, and z are respectively 0.5≦a≦1.25, 0≦x≦1, 10⁻⁶≦y≦2×10⁻², and 0<z≦10⁻²) described in J-A No. S57-23673;

phosphors represented by the composition formula of (Ba₁₋ₓ, M^{II}ₓ)F₂·aBaX₂:yEu, zA (wherein: M^{II} is at least one of beryllium, magnesium, calcium, strontium, zinc, and cadmium; X is at least one of chlorine, bromine, and iodine; A is at least one of arsenic and silicon; and a, x, y, and z are respectively 0.5≦a≦1.25, 0≦x≦10⁻⁶≦y≦2×10⁻¹, and 0<z≦5×10⁻¹) described in JP-A No. 57-23675;

phosphors represented by the composition formula of M^{III}OX:xCe (wherein: M^{III} is at least one of trivalent metal selected from a group consisting of Pr, Nd, Pm, Sm, Eu, Tb, Dy, Ho, Er, Tm, Yb, and Bi; X is either Cl or Br, or both of them; and x is 0<x<0.1) described in JP-A No. 58-69281;

phosphors represented by the composition formula of Ba₁₋ₓM_{x/2}L_{x/2}Fx:yEu²⁺(wherein: M represents at least one of alkali metal selected from a group consisting of Li, Na, K, Rb, and Cs; L represents at least one of trivalent metal selected from a group consisting of Sc, Y, La, Ce, Pr, Nd, Pm, Sm, Gd, Th, Dy, Ho, Er, Tm, Yb, Lu, Al, Ga, In, and Tl; X represents at least one of halogen selected from a group consisting of Cl,Br and I; x is 10⁻²≦x≦0.5; and y is 0<y≦0.1) described in JP-A No. 58-206678;

phosphors represented by the composition formula of BaFX·xA:yEu²⁺(wherein: X is at least one of halogen selected from a group consisting of Cl, Br, and I; A is a burned product of tetrafluoroborate compound; and x is 10⁻⁶≦x≦0.1, and y is 0<y≦0.1) described in JP-A No. 59-27980;

phosphors represented by the composition formula of BaFX·xA:yEu²⁺(wherein: X is at least one of halogen selected from a group consisting of Cl, Br, and I; A is a burned product of at least one of compound selected from a hexafluoro compound group consisting of monovalent or divalent metal salt of hexafluorosilicic acid, hexafluorotitanic acid, and hexafluorozirconic acid; x is 10⁻⁶≦x≦0.1; and y is 0<y≦0.1) described in JP-A No. 59-47289;

phosphors represented by the composition formula of BaFX·xNaX′:aEu²⁺(wherein: X and X′ are respectively at least one of Cl, Br, and I; and x and a are respectively 0<x≦2 and 0<a≦0.2) described in JP-A No. 59-56479;

phosphors represented by the composition formula of M^{II}FX·xNaX′:yEu²⁺:zA (wherein: M^{II}, is at least one of alkaline earth metal selected from a group consisting of Ba, Sr, and Ca; X and X′ are respectively at least one of halogen selected from a group consisting of Cl, Br, and I; A is at least one of transition metal selected from V, Cr, Mn, Fe, Co, and Ni; x is 0<x≦2, y is 0<y≦0.2; and z is 0<z≦10⁻²) described in JP-A No. 59-56480;

phosphors represented by the composition formula of M^{II}FX·aM^{I}X′·bM′^{II}X″₂·cM^{III}X₃·xA:yEu²⁺(wherein: M′_{II} is at least one of alkaline earth metal selected from a group consisting of Ba, Sr, and Ca; M^{I} is at least one of alkali metal selected from a group consisting of Li, Na, K, Rb, and Cs; M′^{II} is at least one of divalent metal selected from a group consisting of Be and Mg; M^{III} is at least one of trivalent metal selected from a group consisting of Al, Ga, In, and Tl; A is a metal oxide; X is at least one of halogen selected from a group consisting of Cl, Br, and I; X′, X″, and X are at least one of halogen selected from a group consisting of F, Cl, Br, and I; a is 0≦a≦2, b is 0≦b≦10⁻², c is 0≦c≦10⁻², and a+b+c≧10⁻⁶; x is 0<x≦0.5; and y is 0<y≦0.2) described in JP-A No. 59-75200;

stimulable phosphors represented by the composition formula of M^{II}X₂·aM^{II}X′₂:xEu²⁺(wherein M^{II} is at least one of alkaline earth metal selected from a group consisting of Ba, Sr, and Ca; X and X′ are at least one of halogen selected from a group consisting of Cl, Br, and I, and X≠X′; a is 0.1≦a≦10.0; and x is 0<x≦0.2) described in JP-A No. 60-84381;

stimulable phosphors represented by the composition formula of M^{II}FX·aM^{I}X′:xEu²⁺ (wherein: M^{II} is at least one of alkaline earth metal selected from a group consisting of Ba, Sr, and Ca; M^{I} is at least one of alkali metal selected from a group consisting of Rb and Cs; X is at least one of halogen selected from a group consisting of Cl, Br, and I; X′is at least one of halogen selected from a group consisting of F, Cl, Br, and I; and a and x are respectively 0≧a≦4.0 and 0≦x≦0.2) described in JP-A No. 60-101173;

stimulable phosphors represented by the composition formula of M^{I}X:xBi (wherein: M^{I} is at least one of alkali metal selected from a group consisting of Rb and Cs; X is at least one of halogen selected from a group consisting of Cl, Br, and I; and x is a numerical value within a range of 0<x≦0.2) described in JP-A No. 62-25189; and

cerium-activated rare earth oxyhalide phosphors represented by LnOX:xCe (wherein: Ln is at least one of La, Y, Gd, and Lu; X is at least one of Cl, Br, and I; x is 0<x≦0.2; the ratio of X to Ln is 0.500<X/Ln≦0.998 in atom ratio; and the maximum wavelength λ of the stimulable exciton spectrum is 550 nm<λX<700 nm) described in JP-A No. 2-229882.

Moreover, M^{II}X₂·aM^{II}X′₂:xEu²⁺stimulable phosphors described in the JP-A No. 60-84381 may contain additives as shown below.

That is, bM^{I}X″ (wherein: M^{I} is at least one of alkali metal selected from a group consisting of Rb and Cs; X″ is at least one of halogen selected from a group consisting of F, Cl, Br, and I; and b is 0<b≦10.0) described in JP-A No. 60-166379; bKX″·cMgX₂·dM^{III}X′₃ (wherein: M^{III} is at least one of trivalent metal selected from a group consisting of Sc, Y, La, Gd, and Lu; X″, X, and X′ are all at least one of halogen selected from a group consisting of F, Cl, Br, and I; and b, c, and d are respectively 0≦b≦2.0, 0≦c≦2.0, 0≦d≦2.0, and 2×10⁻⁵≦b+c+d) described in JP-A No. 60-221483; yB (wherein y is 2×10⁴≦y≦2×10⁻⁴) described in JP-A No. 60-228592; bA (wherein: A is at least one of oxide selected from a group consisting of SiO₂ and P₂O₅; and b is 10⁻⁴≦b≦2×10⁻¹) described in JP-A No. 60-228593; bSiO (wherein b is 0<b≦3×10⁻²) described in JP-A No. 61-120883; bSnX″₂ (wherein: X″ is at least one of halogen selected from a group consisting of F, Cl, Br, and I; and b is 0<b≦10⁻³) described in JP-A No. 61-120885; bCsX″·cSnX₂ (wherein: X″ and X are respectively at least one of halogen selected from a group consisting of F, Cl, Br, and I; and b and c are respectively 0<b≦10.0 and 10⁻⁶≦c≦2×10⁻²) described in JP-A No. 61-235486; and bCsX″ ·yLn³⁺ (wherein: X″ is at least one of halogen selected from a group consisting of F, Cl, Br, and I; Ln is at least one of rare earth selected from a group consisting of Sc, Y, Ce, Pr, Nd, Sm, Gd, Tb, Dy, Ho, Er, Tm, Yb, and Lu; and b and y are respectively 0<b≦10.0 and 10⁻⁶≦y≦1.8×10⁻¹) described in JP-A No. 61-235487.

Among the above stimulable phosphors, divalent europium-activated alkaline earth metal fluorohalide phosphors (such as BaFI:Eu), europium-activated alkali metal halide phosphors (such as CsBr:Eu), iodine-containing divalent europium-activated alkaline earth metal halide phosphors, iodine-containing rare earth element-activated rare earth oxyhalide phosphors, and iodine-containing bismuth-activated alkali metal halide phosphors can be preferably used since they show a high stimulable luminescent brightness.

(Protective Layer)

For the protective layer formed on the phosphor layer, there may be used: a layer formed such that a solution that has been prepared by dissolving a transparent organic high molecular material such as cellulose derivative and polymethyl methacrylate in an appropriate solvent, is coated on the phosphor layer; a sheet for forming a protective film such as a transparent glass plate or an organic high molecular film of polyethylene terephthalate and the like that is separately formed, and provided on the surface of the phosphor layer using an appropriate adhesive; or a film of an inorganic compound formed on the phosphor layer by means of deposition or the like.

Moreover, it may be a protective layer formed from a coated film of an organic solvent-soluble fluororesin, having fine particles such as perfluoroolefine resin powder, silicone resin powder, and TiO₂ particles dispersed and contained therein.

As described above, in order to keep the thermal shrinkage rate (JISC2151, at 150° C. for 30 minutes) of the protective layer 1% or less, there is preferably employed a material that has been previously treated by heat annealing, having a high Tg (glass transition temperature: JIS K7121(1987)). Moreover, preferably a heat treatment of 60° C. or more, is applied at least either before or at the time of its formation.

Hereunder, exemplary aspects of the present invention are enumerated.
(1) A first aspect is an image reading apparatus comprising a disinfection system which applies a disinfection treatment to a radiation image conversion panel, a radiation image conversion film, and/or a light shielding bag that can be used to wrap such a radiation image conversion panel or radiation image conversion film therein.
(2) A second aspect is an image reading apparatus according to the above (1), wherein the disinfection treatment is at least one of a heat treatment, an ultraviolet irradiation treatment, a chemical application treatment, or a gas treatment.
(3) A third aspect is an image reading apparatus according to the above (2), wherein the heat treatment is heating the radiation image conversion panel at 60° C. to 200° C. for 1 second to 10 minutes.
(4) A fourth aspect is an image reading apparatus according to the above (1), wherein the disinfection treatment is a heat treatment, and the disinfection system comprises a temperature control unit.
(5) A fifth aspect is an image reading apparatus according to any one of the above (2) to (4), wherein the disinfection treatment is a heat treatment, and the heat treatment is performed by at least either one of an infrared heater or a far infrared heater.
(6) A sixth aspect is an image reading apparatus according to any one of the above (1) to (5), wherein the disinfection treatment by the disinfection system is performed after radiation image information is erased.
(7) A seventh aspect is an image reading apparatus according to the above (2), wherein the disinfection treatment by the disinfection system is performed by ultra violet irradiation treatment, and the irradiated energy of the ultraviolet irradiation in the ultraviolet irradiation treatment is 0.04 J/cm² or above.
(8) An eighth aspect is a disinfection system for a radiation image conversion panel, a radiation image conversion film, and/or a light shielding bag that can be used to wrap such a radiation image conversion film therein according to the above (7), comprising a disinfection unit which applies a disinfection treatment to any of the radiation image conversion panel a radiation image conversion film, and/or a light shielding bag that can be used to wrap such a radiation image conversion film therein.
(9) An ninth aspect is a disinfection system for a radiation image conversion panel, a radiation image conversion film, and/or a light shielding bag that can be used to wrap such a radiation image conversion film therein according to the above (8), wherein the disinfection treatment by the disinfection unit is at least one of a heat treatment, an ultraviolet irradiation treatment, a chemical application treatment, or a gas treatment.
(10) A tenth aspect is a disinfection system for a radiation image conversion panel, a radiation image conversion film, and/or a light shielding bag that can be used to wrap such a radiation image conversion film therein according to the above (9), wherein the heat treatment is a treatment for heating the radiation image conversion panel at 60° C. to 200° C. for 1 second to 10 minutes.
(11) An eleventh aspect is a disinfection system for a radiation image conversion panel, a radiation image conversion film, and/or a light shielding bag that can be used to wrap such a radiation image conversion film therein according to any one of the above (8) to (10), wherein the radiation image conversion panel has a protective layer, and a thermal shrinkage rate (JISC2151, at 150° C. for 30 minutes) of the protective layer is 1% or less.
(12) A twelfth aspect is a disinfection system for a radiation image conversion panel, a radiation image conversion film, and/or a light shielding bag that can be used to wrap such a radiation image conversion film therein according to the above (11), wherein the protective layer of the radiation image conversion panel is subjected to a heat treatment of 60° C. or more, at least either before or at the time of its formation.
(13) A thirteenth aspect is a disinfection system for a radiation image conversion panel, a radiation image conversion film using a stimulable phosphor, and/or a light shielding bag that can be used to wrap such a radiation image conversion film according to any one of the above (9) to (12), wherein the disinfection treatment by the disinfection unit is heat treatment, and the disinfection unit comprises a temperature control unit.
(14) A fourteenth aspect is a disinfection system for a radiation image conversion panel, a radiation image conversion film, and/or a light shielding bag that can be used to wrap such a radiation image conversion film therein according to any one of the above (8) to (13), wherein further comprises an image reading unit which reads an image on the radiation image conversion panel.
(15) A fifteenth aspect is a disinfection system for a radiation image conversion panel, a radiation image conversion film, and/or a light shielding bag that can be used to wrap such a radiation image conversion film therein according to any one of the above (9) to (14), wherein the disinfection treatment by the disinfection unit is a heat treatment, and the heat treatment is performed by at least either one of an infrared heater or a far infrared heater.
(16) A sixteenth aspect is a disinfection system for a radiation image conversion panel, a radiation image conversion film, and/or a light shielding bag that can be used to wrap such a radiation image conversion film therein according to the above (8), wherein the disinfection treatment by the disinfection system is performed by ultra violet irradiation treatment, and the irradiated energy of the ultraviolet irradiation in the ultraviolet irradiation treatment is 0.04 J/cm² or above.

### EXAMPLES

### Example 1

(Formation of Interlayer)

3400 g of soft acrylic resin (trade name: CRISCOAT P-1018GS manufactured by Dainippon Ink and Chemicals, Incorporated (21% toluene solution)) as a binder and 120 g of phthalic acid ester (trade name: #10 manufactured by Daihachi Chemical Industry Co., Ltd.) as a plasticizer were added and mixed in 3600 g of methyl ethyl ketone, and then dispersed and dissolved using a disper to prepare a dispersion solution for forming an interlayer (viscosity 0.6Pa·s(20° C.)).

A conductive agent and a coloring agent were used which were dispersed by a ball mill in the solution to which a resin had been previously added. This dispersion solution for forming an interlayer was evenly coated on a support (carbon-kneaded polyethylene terephthalate, trade name: X-30 manufactured by Toray Industries, Inc., thickness: 188 μm) to form a coated layer, and was then dried. By so doing, an interlayer having a thickness of 20 μm was formed.

(Production of Phosphor Sheet)

A phosphor sheet to become a phosphor layer was produced as follows. Firstly, as a coating solution for forming a phosphor sheet, 1000 g of phosphor (BaFBr_{0.85}I_{0.15}:Eu²⁺, median diameter 3.5 μm), 36 g of polyurethane elastomer (trade name: PANDEX T5265H (solid)) manufactured by Dainippon Ink and Chemicals, Incorporated) serving as a binder, 4 g of polyisocyanate (trade name: CORONATE HX (solid content 100%) manufactured by Nippon Polyurethane Industry Co., Ltd.) serving as a crosslinking agent, 10 g of epoxy resin (trade name: EPICOAT 1001 (solid) manufactured by Yuka Shell Epoxy Co., Ltd.) serving as an anti-yellowing agent, and 2 g of ultramarine (trade name: SM-1 manufactured by Daiishikasei Co., Ltd.) serving as a coloring agent were added into 120 g of mixed solvent of methyl ethyl ketone and butyl acetate (methyl ethyl ketone/ibutyl acetate (mass ratio)=6/4), and then dispersed using a disper at a blade rotation speed of 2500 rpm for 1 hour to prepare a coating solution having a viscosity of 4.0 Pa·s (25° C.). A coloring agent was used which was dispersed by a ball mill in the solvent to which a resin had been previously added.

This coating solution was evenly coated on a temporary support (polyethylene terephthalate coated with a silicone release material, thickness: 180 μm)) and dried. Then, it was peeled off from the temporary support to produce a phosphor sheet (thickness 150 μm).

(Formation of Phosphor Layer)

Next, the face of the phosphor sheet from which the temporary support was peeled off, was superposed on the interlayer using a calender roll by a continuous compression operation under a pressure of 60MPa, at a roll temperature of 50° C., and at a feed speed of 1.0 m/min. By this heat compression, the phosphor sheet was completely adhered onto the support through the interlayer, and the phosphor layer was formed on the support.

(Formation of Protective Layer)

A PET film having a thickness of 6 μm and a PET film having a thickness of 50 μm were adhered to each other through a repealable adhesive layer, then heat treated at 100° C. The PET film having a thickness of 6 μm was peeled off, and one face thereof was coated with an unsaturated polyester resin solution (trade name: VYLON 30SS manufactured by Toyobo Co., Ltd.), and then dried at 80° C. to provide an adhesive layer. The PET film was adhered onto the phosphor layer through the adhesive layer, to form a protective layer.

Next, this sheet was blanked into an appropriate size (square of 3 cm×3 cm) by a blanking blade (male blade and female blade). Then, a resin (DIAROMER SP3023: EP1004: X-22-2809: CROSSNATE D70=900: 8:2:30 dissolved in MEK) was coated on the surface of the protective layer at the periphery of the blanked sheet with a width of 0.5 to 1 mm extending inward, and then dried (at 50° C.) to produce a radiation image conversion panel.

### Example 2

A protective layer was formed in the same manner as that of Example 1 except that one face of a PET film having a thickness of 9 μm was coated with an unsaturated polyester resin solution (trade name: VYLON 30SS manufactured by Toyobo Co., Ltd.), and heat treated at 80° C. to provide the adhesive layer, to produce a radiation image conversion panel.

### Example 3

A protective layer was formed in the same manner as that of Example 1 except that one face of a PET film having a thickness of 6 μm was coated with an unsaturated polyester resin solution (trade name: VYLON 30SS manufactured by Toyobo Co., Ltd.), and dried at 50° C. to provide the adhesive layer, to produce a radiation image conversion panel.

### Comparative Example 1

A protective layer was formed in the same manner as that of Example 1 except that one face of a PET film having a thickness of 9 μm was coated with an unsaturated polyester resin solution (trade name: VYLON 30SS manufactured by Toyobo Co., Ltd.), and heat treated at 80° C. to provide the adhesive layer, to produce a radiation image conversion panel.

[Measurement of Shrinkage Rate of Protective Layer]

The shrinkage rate of the protective layer on the radiation image conversion panel was measured based on JISC2151 (at 150° C. for 30 minutes). The results are shown in Table 1 below.

[Evaluation of Disinfection Treatment]

The same amount of MRSA was adhered onto each protective layer of the radiation image conversion panels of Examples 1 to 3 and Comparative Example 1. The MRSA was cultured by agar plate cultivation, and then adhered onto the protective layer of the radiation image conversion panel using a brush.

Each radiation image conversion panel was introduced into a scanner with the MRSA adhered thereto, and the radiographic image was read out. Then, while the image was being erased by photoirradiation by self conveyance, a disinfection treatment by heat treatment was performed on the surface of the radiation image conversion panel by an infrared heater (250 W) at a temperature and for a time as shown in Table 1 below. Then, the radiation image conversion panel was taken out from the disinfection apparatus (disinfection unit) by self conveyance, and the remaining MRSA adhered onto the surface of the protective layer was measured. Furthermore, the deformation state of the radiation image conversion panel was visually confirmed. These results are shown in Table 1 below.

The measurement of the surface temperature of the radiation image conversion panel at the time of disinfection treatment and heat control were performed as follows. Firstly, the surface of the radiation image conversion panel was brought into contact with a thermocouple to measure the temperature, and a radiation thermometer at that time was calibrated. Next, the radiation thermometer was covered so as to avoid exposure, and then the surface of the radiation image conversion panel was irradiated by the infrared heater (250 W). The surface temperature of the radiation image conversion panel was measured from the reading and the calibration factor of the radiation thermometer at that time. By feeding back the surface temperature of the radiation image conversion panel, the infrared heater was turned ON/OFF to control the surface temperature of the radiation image conversion panel.

TABLE 1

| | Shrinkage rate of | | | | |
|---|---|---|---|---|---|
| | protective layer on | | | | |
| | radiation image | Disinfection conditions | | | |
| | conversion panel | Temperature | Time | | Shape |
| | (%) | (° C.) | (seconds) | MRSA | deformation |
| Example 1 | 0.3 | 120 | 5 | Killed | None |
| Example 2 | 0.7 | 90 | 10 | Killed | None |
| Example 3 | 1.5 | 110 | 10 | Killed | Slightly |
| | | | | | deformed |
| Comparative | 0.5 | 25 | 10 | No change | None |
| Example 1 | | | | | |

According to Table 1, MRSA remained in the radiation image conversion panel of Comparative Example 1 on which no disinfection treatment by heat treatment was performed. On the other hand, in the radiation image conversion panels of Examples 1 to 3, the MRSA were killed, and no shape deformation of a degree that would be a practical problem was observed. In particular, in the cases of Example 1 and Example 2 where the heat treatment was applied before the protective layer was formed, no shape deformation was observed at all.

### Example 4

A radiation image conversion panel Example 4 was made in the same way as Example 1. The radiation image was read out in the state of having MRSA applied to the light shielding bag. After this it was introduced into the device illustrated in FIG. 3, and after reading out of the radiation image, disinfection treatment was carried out using the heater. Then, after the erasing processing had been carried out of the radiation image information by the erasing unit 39, the amount of MRSA, remaining on the surface of the radiation image conversion panel was investigated. Further, the condition of deformation of the radiation image conversion panel was checked by visual inspection. The result was that the remaining MRSA and condition of deformation were both found to be of the same good condition seen in Example 1.

### Example 5

Example 5 was the same as Example 4, except in that disinfection treatment and erasing processing of the radiation image information by the erasing unit 39 was carried out at one time on the radiation image conversion panel (with MRSA applied thereto) that had been loaded in the device of FIG. 4. The remaining MRSA on the surface of the radiation image conversion panel and condition of deformation of the radiation image conversion panel were checked. The result obtained was that both were found to be of the same good condition seen in Example 1.

### Example 6

The Example 6 was the same as Example 4, except in that disinfection treatment using a heater was carried out in the device of FIG. 5 after erasing processing of the radiation image information by the erasing unit 39. The remaining MRSA on the surface of the radiation image conversion panel and condition of deformation of the radiation image conversion panel were checked. The result obtained was that both were found to be of the same good condition seen in Example 1.

According to the present invention, there can be provided a disinfection system for a radiation image conversion panel which can evenly and efficiently disinfect, a radiation image conversion panel disinfected by the disinfection system, and an image reading apparatus comprising the disinfection system.

All publications, patent applications, and technical standards mentioned in this specification are herein incorporated by reference to the same extent as if each individual publication, patent application, or technical standard was specifically and individually indicated to be incorporated by reference.

What is claimed is:

1. An image reading apparatus comprising a disinfection system which applies a disinfection treatment to a radiation image conversion panel, a radiation image conversion film, and/or a light shielding bag that can be used to wrap such a radiation image conversion panel or radiation image conversion film therein.

2. The image reading apparatus according to claim 1, wherein the disinfection treatment is at least one of a heat treatment, an ultraviolet irradiation treatment, a gas treatment, or a chemical application treatment.

3. The image reading apparatus according to claim 2, wherein the heat treatment is heating the radiation image conversion panel at 60° C. to 200° C. for 1 second to 10 minutes.

4. The image reading apparatus according to claim 1, wherein the disinfection treatment is a heat treatment, and the disinfection system comprises a temperature control unit.

5. The image reading apparatus according to claim 2, wherein the disinfection treatment is a heat treatment, and the heat treatment is performed by at least either one of an infrared heater or a far infrared heater.

6. The image reading apparatus according to claim 1, wherein the disinfection treatment by the disinfection system is performed after radiation image information is erased.

7. The image reading apparatus according to claim 2, wherein the disinfection treatment by the disinfection system is performed by ultra violet irradiation treatment, and the irradiated energy of the ultraviolet irradiation in the ultraviolet irradiation treatment is 0.04 J/cm² or above.

8. A disinfection system for a radiation image conversion panel, a radiation image conversion film, and/or a light shielding bag that can be used to wrap such a radiation image conversion panel or radiation image conversion film therein comprising a disinfection unit which applies a disinfection treatment to any of the radiation image conversion panel, a radiation image conversion film, and/or a light shielding bag that can be used to wrap such a radiation image conversion film therein.

9. The disinfection system for a radiation image conversion panel, a radiation image conversion film, and/or a light shielding bag that can be used to wrap such a radiation image conversion film therein according to claim 7, wherein the disinfection treatment by the disinfection unit is at least one of a heat treatment, an ultraviolet irradiation treatment, a chemical application treatment, or a gas treatment.

10. The disinfection system for a radiation image conversion panel, a radiation image conversion film, and/or a light shielding bag that can be used to wrap such a radiation image conversion film therein according to claim 9, wherein the heat treatment is a treatment for heating the radiation image conversion panel at 60° C. to 200° C. for 1 second to 10 minutes.

11. The disinfection system for a radiation image conversion panel, a radiation image conversion film, and/or a light shielding bag that can be used to wrap such a radiation image conversion film therein according to claim 10, wherein the radiation image conversion panel has a protective layer, and a thermal shrinkage rate of the protective layer under a condition of 150° C. for 30 minutes is 1% or less.

12. The disinfection system for a radiation image conversion panel, a radiation image conversion film, and/or a light shielding bag that can be used to wrap such a radiation image conversion film therein according to claim 11, wherein the protective layer of the radiation image conversion panel is subjected to a heat treatment of 60° C. or more, at least either before or at the time of its formation.

13. The disinfection system for a radiation image conversion panel, a radiation image conversion film, and/or a light shielding bag that can be used to wrap such a radiation image conversion film therein according to claim 9, wherein the disinfection treatment by the disinfection unit is a heat treatment, and the disinfection unit comprises a temperature control unit.

14. The disinfection system for a radiation image conversion panel, a radiation image conversion film, and/or a light shielding bag that can be used to wrap such a radiation image conversion film therein according to claim 8, further comprising an image reading unit which reads an image on the radiation image conversion panel.

15. The disinfection system for a radiation image conversion panel, a radiation image conversion film, and/or a light shielding bag that can be used to wrap such a radiation image conversion film therein according to claim 9, wherein the disinfection treatment by the disinfection unit is a heat treatment, and the heat treatment is performed by at least either one of an infrared heater or a far infrared heater.

16. The disinfection system for a radiation image conversion panel, a radiation image conversion film, and/or a light shielding bag that can be used to wrap such a radiation image conversion film therein according to claim 8, wherein the disinfection treatment by the disinfection system is performed by ultra violet irradiation treatment, and the irradiated energy of the ultraviolet irradiation in the ultraviolet irradiation treatment is 0.04 J/cm² or above.
